(19) United States
(12) Patent Application Publication
(10) Pub. No.: US 2012/0150122 A1
(43) Pub. Date: Jun. 14, 2012

(54) Protective bandaging for point of insertion of shunt tubing

(76) Inventor: Beverly Harper, Decatur, GA (US)

(21) Appl. No.: 12/928,425

(22) Filed: Dec. 10, 2010

(51) Int. Cl. A61M 25/02

(52) U.S. Cl. 604/180

(57) ABSTRACT

Disclosed is a bandage pad of any of a variety of geometric shapes, used to cover a surgically-incised body area wherein shunt tubing, a catheter, or similar device protrudes from the body area. The bandage pad protects the subject area from infection which conceivably could be caused by contact with soapy, and/or contaminated water while the patient is showering or bathing. The bandage pad comprises a pliable, rigid, or semi-rigid shell-like cover having a convex outer surface, with an absorbent, cushioned material permanently attached to the inner surface of the shell. A continuous flange forms the outer edge of the shell-like cover, said flange having an adhesive-coated material covering its inner surface. The adhesive-coated material thereby enables adherence of the bandage pad to a patient's skin.

### REFERENCE TO RELATED APPLICATION

Not applicable.

### STATEMENT REGARDING FEDERALLY SPONSORED RESEARCH OR DEVELOPMENT

Not applicable.

### NAMES OF THE PARTIES TO A JOINT RESEARCH AGREEMENT

Not applicable.

### BACKGROUND OF THE INVENTION

(1) Field of the Invention

The inventive concept presented herein generally is concerned with protective medical bandages which afford protection to a surgical site on a human body. A bandage is commonly thought of as a segment of material used either to support a medical device such as a dressing or splint, or to provide support to a body part during the healing process. Bandages come in a variety of types, designs, and sizes, from generic cloth strips to specially-shaped devices for a specific wound or the incisions remaining in the vicinity of a surgical area. The present inventive concept is in the class of bandages for holding, covering, or stabilizing a medical device such as a shunt or associated tubing.

(2) Description of the Related Art

Prior inventions an disclosed devices reveal many types of bandages which function similarly to the present inventive concept.

In U.S. Pat. No. 7,605,299 (2009), the inventor devised a disposable bandage for covering a wound area of a skin surface, including a self-adhesive first fastener portion and a second fastener portion, a dome having a generally rectilinear shape and constructed from a thin rigid material, the dome being coupled to the first fastener portion along one edge and coupled to the second fastener portion along a second edge. A gasket, formed of a stretchable material, extends around the underside of the dome, which gasket is held in contact with the skin surface when the first fastener portion and the second fastener portion are pressed to a user's skin.

US patent application publication #2008/0103451 (2008) reveals kits and bandages that accommodate a medical device extending from an incision in a patient. The bandage includes an outer surface, inner absorbent layer and one or more closure mechanisms. The outer surface includes a slit extending from an outer peripheral edge to an inner peripheral edge that forms an aperture to accommodate a medical device.

The inventor in US patent application publication 2008/0033377 devised an adhesively attachable bandage for an open wound. A one-way valve permits air or other fluids to move through the bandage in one direction, as to escape from a body cavity, but prevents fluids from moving in the other direction, as in entering the wound. A selectively sealable port can be opened to give direct access to the wound.

US patent application publication 2004/0215118 discloses a disposable, water resistant plastic surgical bandage, similar to a cast, having elastic bands at any open end, and powder rings inside at all opens ends, said powder rings turning to gel on contact with any moisture. The bandage may be patterned for the head, neck, chest, waist, arm, leg, and foot to protect against water seepage when taking a shower or bath.

A bandage pad comprising a pad in an approximate-quadrangular portion and approximate-arched portions attached to both lateral sides of the approximate-quadrangular portion and all the periphery of said pad being sealed with ultrasonic treatment is disclosed in U.S. Pat. No. 6,743,964 (2004). The pad prevents peeling-off from four corners thereof and intrusion of water.

In U.S. Pat. No. 6,528,697 (2003), a modular bandage for treating wounds located on highly contoured, non-planar regions of a person includes a stand-off for placement near the wound, and a film of material for forming a sealed environment over the wound in conjunction with the standoff. A film of material for such a bandage may include various uniform or compound structures.

U.S. Pat. No. 5,707,348 (1998), discloses an intravenous bandage for securing an intravenous administration tube to the epidermis. A preformed bandage includes a pocket in which a bended portion of an administration tube is held in secured capture. One embodiment includes an umbilicus member which is incorporated for attachment of the administration tube to the epidermis.

U.S. Pat. No. 4,633,863 (1987), has an arterial anchor bandage with a transparent bulbous shield which protrudes above a tri-lobed outer covering layer. The bulbous shield has an opening sized to receive the tube from an arterial catheter and has a generally elliptical cross section. An absorbent layer between the shield and the skin surface provides a wicking function to remove fluids from the area where the arterial catheter enters the patient.

### BRIEF SUMMARY OF THE INVENTIVE CONCEPT

The inventive concept herein discloses a bandage pad of any of a variety of geometric shapes, which is designed to cover a surgically-incised body area wherein a y-shaped tubing, shunt, catheter, or similar device protrudes from the body area. For illustrative purposes only, the bandage pad disclosed in the present inventive concept is utilized to cover shunt tubing commonly protruding from the affected area of dialysis patients. The primary purpose of the bandage pad is to protect the subject area from infection which conceivably could be caused by contact with soapy, and/or contaminated water while the patient is showering or bathing. The bandage pad comprises a rigid or semi-rigid shell with a perimetral lip having an adhesive surface covering the inner surface of said lip and a non-stick absorbent cushion material permanently attached to the inner surface of the shell.

### BRIEF DESCRIPTION OF THE VIEWS OF THE DRAWINGS

The objects, features, and advantages of the concept presented in this application are more readily understood when referring to the accompanying drawings. The drawings, totaling five figures, show the basic function of the bandage pad. In the several figures, like reference numbers are used in each figure to correspond to the same component as may be depicted in other figures.

FIG. 1 shows a head-on view of the exterior surface of the bandage pad.

FIG. 2 is a cutaway side view of the bandage pad as seen from the line A-A.

FIG. 3 presents an interior view of the bandage pad, also showing the absorbent cushion material.

FIG. 4 illustrates a bandage pad enclosed in its partially-opened packaging container.

FIG. 5 is a view of a patient with the bandage pad adhering to the skin in the vicinity of shunt tubing.

Nomenclature For Invention Components

| | |
|---|---|
| 1. | Bandage pad |
| 2. | Outer cover |
| 3. | Adhesive-coated material |
| 4. | Outer perimetral flange |
| 5. | Inner perimetral flange |
| 6. | Outline of recess of cover |
| 7. | Recess of cover |
| 8. | Absorbent cushion material |
| 9. | Packaged bandage pad |
| 10. | Wide end |
| 11. | Narrow end |
| 12. | Plastic wrapping film |
| 13. | Box container |
| 14. | Shunt tubing |
| 15. | Direction of film opening |
| 16. | Left side dimension |
| 17. | Right side dimension |

### DETAILED DESCRIPTION OF THE INVENTIVE CONCEPT

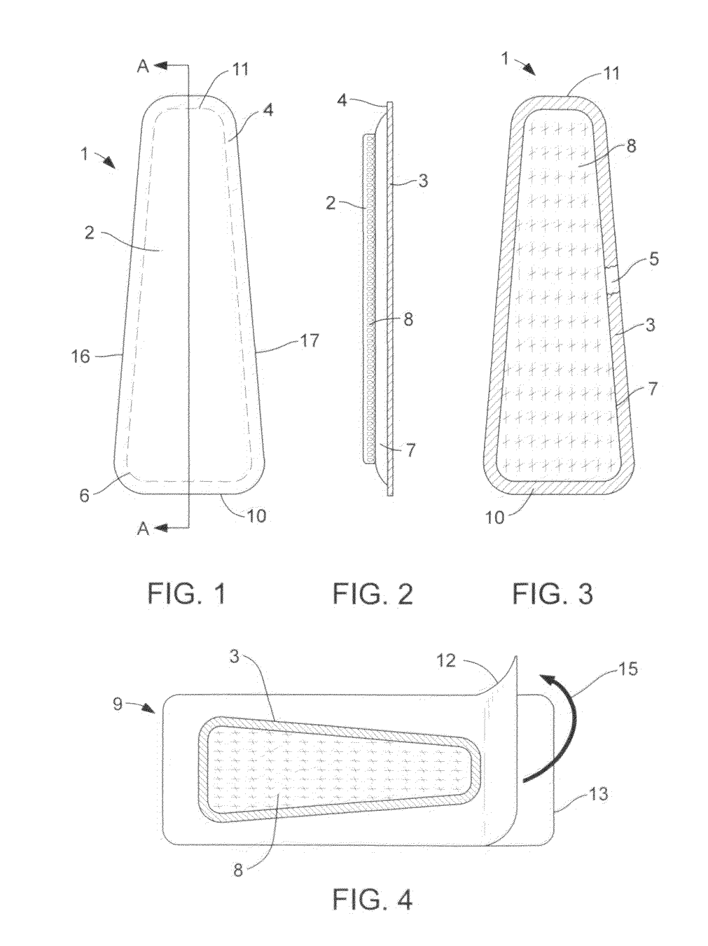

In referring to FIG. 1, there is shown the outer cover 2 of a bandage pad 1 having the general shape of a trapezoid. Also shown is the outer perimetral flange 4 of the cover 2. The trapezoidal shape of the outer cover 2 is fabricated with two equal-length side dimensions 16, 17, a wide end 10, and a narrow end 11. The wide end 10 serves the purpose of accommodating coverage of a Y-junction which is found in certain types of medical tubing, shunts, etc. The outer cover 2 may be constructed from a wide assortment of materials of varying textures and strengths, from pliant, resilient, semi-rigid, and rigid surface characteristics.

In viewing FIG. 2, there is presented a cutaway side view of the bandage pad 1, as seen from the perspective of line A-A of FIG. 1. Shown in FIG. 2 are cross sections of the outer cover 2 and the adhesive-coated material 3, which material is attached to the inside of the outer perimetral flange 4 of the bandage pad 9. The absorbent cushion material 8 abuts the area of the curvature of the recess 7 of the cover 2. The outer cover 2 may be fabricated in any of several different geometric shapes, including circular, elliptical, triangular, square, or other multi-sided figures.

FIG. 3 presents a view of the interior of the bandage pad 1. The absorbent cushion material 8 is shown as being adhesively dispersed onto the entire surface of the inside cover of the bandage pad 1. An adhesive-coated material 3 covers the entirety of the inner perimetral flange 5, an uncovered portion of the inner perimetral flange 5, also being shown.

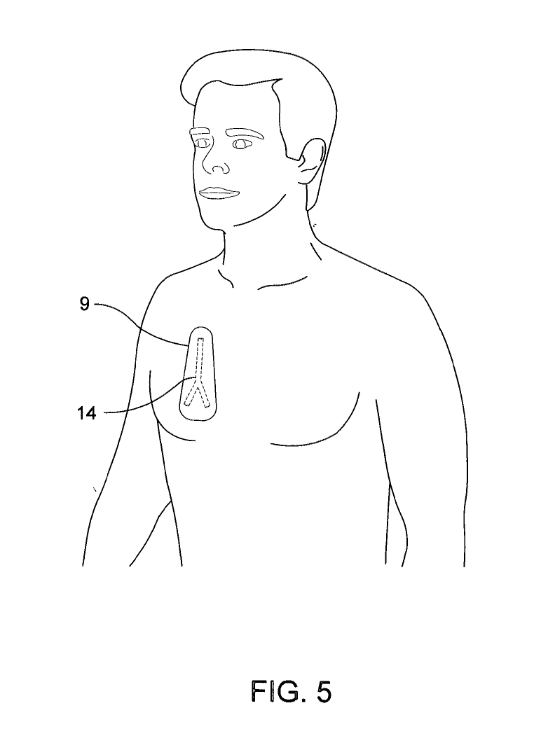

FIG. 4 illustrates a completely packaged bandage pad 9 in the process of being unwrapped in the direction of the arrow 15. The outer cover 2 of the bandage pad 9 is removably imbedded into a form-fitted, contoured box container 13. A thin plastic wrapping film 12 adhesively covers the entire flat surface of the box container 13 and also covers the adhesive-coated material 3 which overlays the inner perimetral flange 5. Once the thin plastic wrapping film 12 is peeled from the box container 13, the bandage pad 1 is ready for use. FIG. 5 depicts a patient having the bandage pad 9 applied to cover a Y-shaped shunt tubing 14.

While preferred embodiments of the present inventive concept have been shown and disclosed herein, it will be obvious to those persons skilled in the art that such embodiments are presented by way of example only and not as a limitation to the scope of the inventive concept. Numerous variations, changes, and substitutions may occur or be suggested to those skilled in the art without departing from the intent, scope, and totality of the inventive concept. Such variations, changes, and substitutions may involve other features which are already known per se and which may be used instead of, or in addition to features already disclosed herein. Accordingly, it is intended that this inventive concept not be limited by the scope of the accompanying claims.

What is claimed is:

1. A bandage pad used for mounting on a patient's skin in the immediate vicinity of a surgical site whereupon shunt tubing, a catheter, or similar medical appliance protrudes from said site, comprising
(a) a shell-like cover having a convex outer surface and a concave inner surface, said inner surface forming a shallow cavity and further, said shell-like cover having a perimeter flange with an inner surface and an outer surface, the perimeter flange extending the entirety of the edge perimeter of said shell-like cover;
(b) a layer of pliant absorbent material; and
(c) a strip of material having an adhesive side and a non-adhesive side;
(d) a sanitized plastic film; wherein
said layer of pliant absorbent material is attached, at a uniform depth, to the inner surface of said shell-like cover; said non-adhesive side of said strip of material is permanently attached to the inner surface of said perimeter flange, thereby exposing the adhesive side of said strip of material, whereupon said adhesive side and said cavity are contemporaneously covered by a sanitized plastic film, thereby forming an airtight seal over said bandage pad.

2. A bandage pad and its protecting packaging, used for mounting on a patient's skin in the immediate vicinity of a surgical site whereupon shunt tubing, a catheter, or similar medical appliance protrudes from said site, comprising
(a) a shell-like cover having a convex outer surface and a concave inner surface, said inner surface forming a shallow cavity and further, said shell-like cover having a perimeter flange with an inner surface and an outer surface, the perimeter flange extending the entirety of the edge perimeter of said shell-like cover;
(b) a layer of pliant absorbent material;
(c) a strip of material having an adhesive side and a non-adhesive side;
(d) a protective, flanged carton having a contoured cavity, and
(e) a sanitized plastic film; and
(f) a wide-flanged carton; wherein
said layer of pliant absorbent material is attached, at a uniform depth, to the inner surface of said shell-like cover, the non-adhesive side of said strip of material is permanently attached to the inner perimeter flange of said shell-like cover, thereby exposing the adhesive side of said strip of material, whereupon said adhesive side and said cavity are contemporaneously covered by said sanitized plastic film, thereby forming an airtight seal over said bandage pad, and further, the entirety of the outer surface of said shell-like cover is enveloped within a protective wide-flanged carton, said carton contoured to accept the outer convex surface of the shell-like cover, thereby forming an integral package.

3. The bandage pad as in claim 1, further having an outer convex surface comprised of a rigid material.

4. The bandage pad as in claim 2, further having an outer convex surface comprised of a rigid material.

5. The bandage pad as in claim 1, further, said shell-like cover having an outer convex surface comprised of a flexible, pliant material.

6. The bandage pad as in claim 2, further, said shell-like cover having an outer convex surface comprised of a flexible, pliant material.

7. A bandage pad and its protecting packaging, used for mounting on a patient's skin in the immediate vicinity of a surgical site whereupon shunt tubing, a catheter, or similar medical appliance protrudes from said site, comprising
(a) a shell-like, vertically-oriented cover having a convex outer surface and a concave inner surface, said inner surface forming a shallow cavity and further, said shell-like cover having a perimeter flange with an inner surface and an outer surface, the perimeter flange extending the entirety of the edge perimeter of said shell-like cover;
(b) said shell-like cover having a trapezoidal shape, with a narrow top end, a wider bottom end, and two upwardly-angled sides, the length of each angled side being approximately triple the dimension of the wider bottom end;
(c) a layer of pliant absorbent material;
(d) a strip of material having an adhesive side and a non-adhesive side;
(e) a protective, flanged carton having a contoured cavity; and
(f) a sanitized plastic film; and
(g) a wide-flanged carton, wherein
said layer of pliant absorbent material is attached, at a uniform depth, to the inner surface of said shell-like cover, the non-adhesive side of said strip of material is permanently attached to the inner perimeter flange of said shell-like cover, thereby exposing the adhesive side of said strip of material, whereupon said adhesive side and said cavity are contemporaneously covered by said sanitized plastic film, thereby forming an airtight seal over said bandage pad, and further, the entirety of the outer surface of said shell-like cover is enveloped within a protective wide-flanged carton, said carton contoured to accept the outer convex surface of the shell-like cover, thereby forming an integral package.

8. The bandage pad as in claim 7, wherein said shell-like cover forms a trapezoidal shape, with a top end of dimension in the range of 1 inch to 1.5 inches, a bottom end of a dimension in the range of 1.5 inches to 3 inches, and two upwardly-angled sides, the length of each angled side being in the range of 4 inches to 6 inches.

9. The bandage pad as in claim 7, wherein said shell-like cover forms a trapezoidal shape, with a top end of dimension in the range of 1.5 inches to 3 inches, a bottom end of a dimension in the range of 3 inches to 5 inches, and two upwardly-angled sides, the length of each angled side being in the range of 6 inches to 8 inches.

10. The bandage pad as in claim 5, further, said shell-like cover having an outer convex surface comprised of a rigid material.

11. The bandage pad as in claim 5, further, said shell-like cover having an outer convex surface comprised of a flexible, pliant material.

12. The bandage pad as in claim 7, further, said shell-like cover having an outer convex surface comprised of a rigid material.

13. The bandage pad as in claim 7, further, said shell-like cover having an outer convex surface comprised of a flexible, pliant material.
